(19) United States
(12) Patent Application Publication
(10) Pub. No.: US 2025/0166800 A1
(43) Pub. Date: May 22, 2025

(54) Homecare System For Facilitating Collaboration

(71) Applicant: AgileRN LLC, Wheaton, IL (US)

(72) Inventors: Joel C. Relova, Jacksonville, FL (US); Rodney G.B. Clements, Jacksonville, FL (US)

(21) Appl. No.: 18/513,477

(22) Filed: Nov. 17, 2023

(51) Int. Cl. G16H 40/20; G16H 10/60; G16H 40/67

(52) U.S. Cl. CPC G16H 40/20; G16H 10/60; G16H 40/67

(57) ABSTRACT

The present disclosure presents a homecare system for facilitating collaboration, which enables multiple homecare agencies and homecare providers to collaborate effectively and efficiently. The homecare system comprises a software application with a plurality of components. A matching component is configured to generate potential matches of homecare providers for each of the patients with specific requirements based on various attributes. A revenue sharing component allows the homecare agencies to collaborate when no suitable matches are available, or the suitable homecare provider is within another homecare agency. Other components including a recruiting component, a mapping component, a referring component, a scheduling component, and a medical record component optimizes the process of sourcing the homecare providers, appointment scheduling, and other administrative task or management.

### FIELD OF THE INVENTION

The subject matter disclosed herein generally relates to a homecare system and more specifically to a homecare system for facilitating collaboration between multiple institutions and professionals.

### BACKGROUND

The advancement of healthcare, pharmaceuticals, and medical technologies have collectively contributed to a notable increase in global life expectancy and aging population. These progressions may cause increased demand for home care services due to a variety of factors. First, elderly individuals may prefer receiving care in the comfort of their own homes rather than in institutional settings such as hospitals or nursing homes, which may also be beneficial and encouraging when the individual is in closer proximity to their family. Secondly, with the advancement of medical technology, remote monitoring and other assistive devices can be furnished at individual homes, which provides a more flexible and personalized environment for the individual. Furthermore, homecare services reduce the cost for overnight hospital stays and the risk of hospital-acquired infections.

However, the homecare system requires a wide range of institutions and professionals with meticulous coordination, which may pose difficulty for collaboration between all care providers. For example, currently, registered nurses or licensed practical nurses are often available either directly from a hospital or a small portion available through agencies representing the nurse, which may present challenges for medical clinics or physician practices to manage the complexities of sourcing nurses to in-home patients. As a result, the management is often outsourced to nursing agencies. Nevertheless, other hurdles arise because most nursing agencies are small businesses which may be insufficient with ideal tools or infrastructure for providing seamless cooperation, communication, or information sharing. Furthermore, in some circumstances, the most suitable nurse for specific requirements of the patient is represented by another agency.

Consequently, with the homecare industry emerging as a rapidly expanding industry, there is a need for a homecare system for facilitating collaboration to provide a system for different nursing agencies, patients, and a variety of professionals including but not limited to nurses, pharmacies, physicians, and insurance companies.

### SUMMARY OF THE INVENTION

The following is a concise summary of the invention presented herein with the primary aim of providing a preliminary understanding of certain aspects of the invention. It should be noted, however, that this summary is not intended to serve as a comprehensive overview of the invention, nor does it seek to identify or describe any critical or significant elements of the invention or the boundaries of its scope. Its sole purpose is to provide a rudimentary understanding of some of the invention's concepts and features, which will be expounded upon in greater detail in the ensuing sections.

A homecare system for facilitating collaboration, hereinafter referred to as the “homecare system,” facilitates effective and efficient collaboration among various medical institutions, streamlines the process of sourcing and matching medical professionals to in-home patients with specific requirements and optimizes appointment scheduling, and other administrative task or management.

The homecare system generally comprises a network, two or more homecare agencies, a plurality of homecare providers, a plurality of patients, a plurality of computing devices associated with each of the two or more homecare agencies and the plurality of homecare providers respectively, one or more servers, one or more monitoring devices, a database, and a software application, which provides a plurality of components, including, but not limited to, a matching component, a revenue sharing component, a recruiting component, a mapping component, a scheduling component and medical record component. The homecare system provides compatible potential matches of the homecare providers for each of the patients and enhances cooperation among the two or more homecare agencies by the matching component and the revenue sharing component, facilitates the sourcing of homecare providers for homecare agencies by the recruiting component, optimizes efficiency of the process of homecare visits by the scheduling component, and optimizes administrative task and management by the medical record component.

Each of the homecare providers may be affiliated with one or more homecare agencies, or not affiliated with any homecare agencies. Each of the two or more homecare agencies has a roster of patients and homecare providers. The affiliation between the homecare agencies and the homecare providers may include employment or contractual connection.

The software application of the homecare system may be accessible via the plurality of computing devices to a user as either as a homecare agency or a homecare provider, upon the user signing into a user account. The homecare agency and the homecare provider may gain access by signing into their respective user accounts, a homecare agency account, and a homecare provider account. The homecare agency can further create, manage, delete, view or designate user sub-user accounts for payroll administrators or office administrators. It is also anticipated that the homecare system may also create user accounts to a patient, a pharmacy, a physician, or other medical professionals.

The one or more monitoring devices are configured to monitor physiological parameters of each of the patients and are connected to the network wirelessly, such that the physiological parameters can be remotely viewed by the plurality of computing devices. The software application provides the two or more homecare agencies and the homecare providers with different levels of access to the physiological parameters of each of the patients. Each of the two or more homecare agencies would only have access to the physiological parameters of the patients within their roster, and each of the homecare providers would only have access to the physiological parameters of the patients matched to them.

In an implementation of the invention, the matching component generates a plurality of potential matches of homecare providers for each of the patients which is determined based on a plurality of attributes. The plurality of attributes comprises dates, times, geographic locations, requirements of each of the patients and qualifications of each of the homecare providers. The potential matches of the homecare providers comprise the homecare providers within the roster or not within the roster of each of the two or more homecare agencies. The matching component determines for each of the two or more homecare agencies, whether the homecare providers of the potential matches are listed on the roster of each of the two or more homecare agencies or listed on the roster of another homecare agency. In a circumstance where the potential matches are the homecare providers listed within the roster, the matching component provides a confirmation option to the homecare agency allowing the homecare agency with the roster to confirm a matched homecare provider for each of the patients. After the matched homecare provider is confirmed, the homecare agency with the roster and the matched homecare provider have access to view the physiological parameters of the patient. In another case where the potential matches are within the roster of another agency, the revenue sharing component allows the homecare care providers within the roster of another agency to accept or reject the patient. When the patient is accepted, the revenue sharing component creates an engagement record including an agreement of division of revenue between the two homecare agencies and provides the two homecare agencies and the homecare provider accepting the patient access to view the physiological parameters of each of the patient.

The revenue sharing component may comprise a collaborative platform. The collaborative platform is configured to allow each of the two or more homecare agencies to list each of the patients within the roster on the collaborative platform and enable each of the two or more homecare agencies to submit a proposal requesting to provide homecare to the patients. The proposal includes suitable homecare providers and corresponding rates for providing the homecare to the patients. After viewing all the proposals, the homecare agency with the roster of the patient may select the homecare agency to collaborate. Thereafter, an engagement record will be generated, including an agreement of division of revenue between the two homecare agencies collaborating and provides the two homecare agencies and the homecare provider collaborating access to view the physiological parameters of each of the patient.

The attached drawings and the detailed description of the preferred embodiments that follow will provide a clearer understanding of these and other objectives, features, and advantages of the present invention.

### BRIEF DESCRIPTION OF THE DRAWINGS

FIG. 1 is a schematic diagram that depicts an exemplary embodiment of a homecare system for facilitating collaboration.

FIG. 2 is a schematic diagram that depicts how the homecare system may be accessed by homecare agencies and homecare providers.

FIG. 3 is a schematic diagram that depicts how the homecare system may be accessed by homecare agencies, homecare providers, patients, pharmacies and physicians.

FIG. 4 depicts each of the homecare agencies having their own roster of patients and homecare providers.

FIG. 5 is a schematic diagram that depicts an exemplary, non-limiting embodiment of a revenue sharing component.

FIG. 6 is a schematic diagram that depicts an alternative exemplary, non-limiting embodiment of a revenue sharing component.

FIG. 7 is a nonlimiting, exemplary graphic page depicting the patients within the roster of one of the homecare agencies.

FIG. 8 is a nonlimiting, exemplary graphic page depicting may perform one or more actions relating to the patients within the roster.

FIG. 9 is a nonlimiting, exemplary graphic page depicting an interactive map.

FIG. 10 is a nonlimiting, exemplary graphic page depicting the revenue sharing component.

FIG. 11 is another nonlimiting, exemplary graphic page depicting the revenue sharing component.

FIG. 12 is another nonlimiting, exemplary graphic page depicting the revenue sharing component.

FIG. 13 is another nonlimiting, exemplary graphic page depicting the revenue sharing component.

FIG. 14 is another nonlimiting, exemplary graphic page depicting the revenue sharing component.

FIG. 15 is a nonlimiting, exemplary graphic page depicting a calendar view provided by a scheduling component.

FIG. 16 is a nonlimiting, exemplary graphic page depicting a medical record component.

FIG. 17 is another nonlimiting, exemplary graphic page depicting a medical record component.

FIG. 18 is a schematic diagram that depicts association between a variety of features provided by an exemplary, non-limiting embodiment of homecare agency account and payroll administrator sub-account dashboard.

FIG. 19 is a schematic diagram that depicts association between a variety of features provided by an exemplary, non-limiting embodiment of a office administrator sub-account dashboard.

FIG. 20 is a schematic diagram that depicts association between a variety of features provided by an exemplary, non-limiting embodiment of a homecare provider dashboard.

### NUMBER REFERENCES

10—Computing device
101—Touch Screen
102—Camera
103—GPS receiver
104—Microphone
105—Internal Memory
11—Homecare Agency
12—Homecare Provider
13—Patient
14—Pharmacy
15—Physician
20—Monitoring Device
30—Software Application
31—Matching Component
32—Revenue Sharing Component
33—Recruiting Component
34—Mapping Component
35—Referring Component
36—Scheduling Component
37—Medical Record Component
40—Server
50—Database

### DETAILED DESCRIPTION OF THE INVENTION

The following detailed description and accompanying drawings provide a comprehensive disclosure of exemplary embodiments for the purpose of facilitating one of ordinary skill in the relevant art to make and use the invention. Therefore, the detailed description and illustration of the one or more exemplary embodiments presented herein are purely exemplary in nature and are not intended to limit the scope of the invention or its protection in any manner. It is further noted that the drawings may not be to scale, and in some cases, certain details may be omitted which are not necessary for an understanding of the present invention, such as conventional details of fabrication and assembly.

The present invention is a homecare system for facilitating collaboration, hereinafter referred to as the “homecare system” 1 that facilitates effective and efficient collaboration among various medical institutions, streamlines the process of sourcing and matching medical professionals to in-home patients with specific requirements and optimizes appointment scheduling, and other administrative task or management. While there is a wide range of medical institutions and medical professionals, this application discusses the invention in the context of collaboration between different homecare agencies and sourcing of homecare providers.

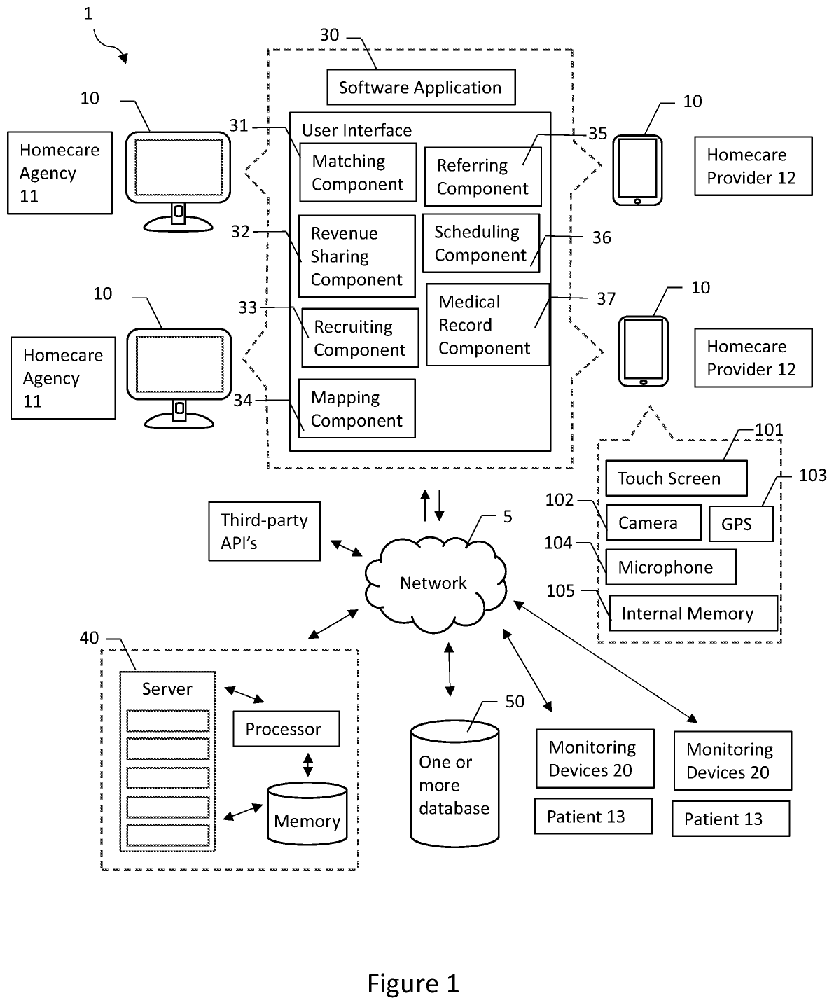

As shown in FIG. 1, the homecare system 1 comprises a network 5, two or more homecare agencies 11, a plurality of homecare providers 12 and patients 13, a plurality of computing devices 10 associated with each of the two or more homecare agencies 11 and each of the homecare providers 12 respectively, one or more servers 40, a database 50, and a software application 30. The software application 30 provides a plurality of components, including a matching component 31, a revenue sharing component 32, a recruiting component 33, a mapping component 34, a referring component 35, a scheduling component 36, and a medical record component 37. In addition, the revenue sharing component 32 may also include a collaborative platform. The plurality of homecare providers 12 further comprises homecare providers 12 affiliated with one of the two or more homecare agencies 11, homecare providers 12 affiliated with more than one of the two or more homecare agencies 11 or homecare providers 12 not affiliated with the two or more homecare agencies 11. Each of the two or more homecare agencies 11 includes a roster of patients 13 referred to them, along with the homecare providers 12 affiliated with each of the two or more homecare agencies 11. The homecare providers 12 may include but are not limited to, registered nurses, licensed practical nurses, or other qualified caregivers. It is anticipated that the plurality of computing devices 10 may include but are not limited to a workstation or a mobile device such as a smart phone, a tablet, a smart watch, or a pair of smart glasses.

Each of the computing devices 10 may comprise a plurality of hardware components, including a touch screen 101, a camera 102, a global positioning system receiver (GPS receiver) 103, a microphone 104, and an internal memory 105. The touchscreen 101 enables the users to execute handwritten signatures on the screen for necessary documents. The camera 102 may capture two-dimensional codes on medication or supplies for each of the patients 13 and identify medical data from the two-dimensional codes. In addition, the camera 102 may facilitate remote video communication between the homecare agencies 11 and the homecare providers 12. The GPS receiver 103 is configured to determine respective locations of the computing devices 10 and denote the respective locations to the network 5. The microphone 104 includes a variety of functions, such as audio recording, voice messaging, sound recognition and communication between the plurality of computing devices 10.

The network 5 has a plurality of end nodes wirelessly connected to it. The one or more servers 40 of the homecare system 1 communicates with the network 5 and stores one or more databases 50. The one or more databases 50 stores data related to the two or more homecare agencies 11, the plurality of homecare providers 12, and the patients 13. The database is accessible through the network 5 and is updated by the software application 30.

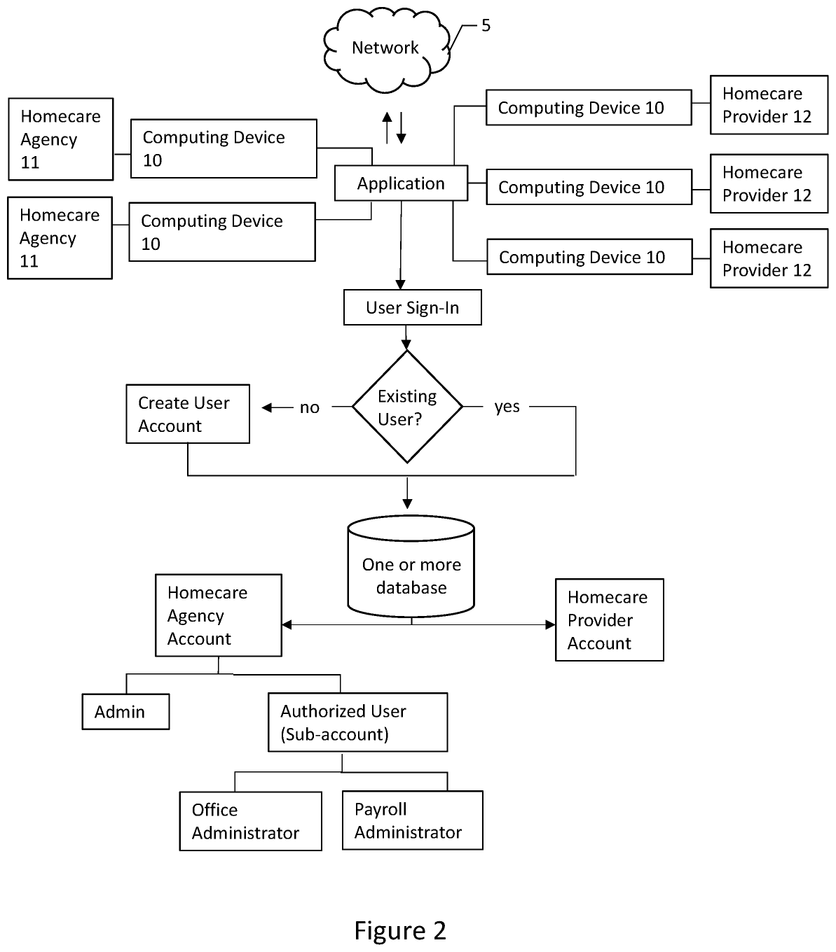

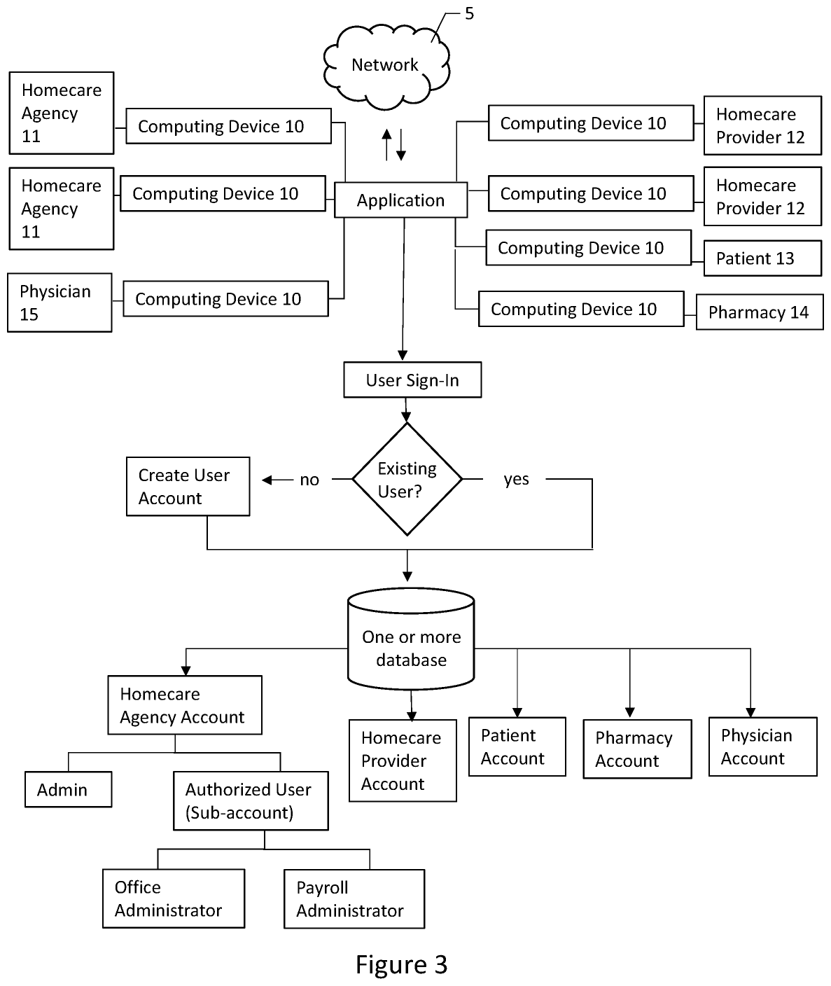

The software application 30 is downloadable and accessible via the plurality of computing devices 10 to a user as either as a homecare agency 11 or a homecare provider 12, upon the user signing into a user account. The homecare agency 11 and the homecare provider 12 may gain access by signing into their respective user accounts, for example a homecare agency account and a homecare provider account, as shown in FIG. 2. The homecare agency 11 can further create, manage, delete, view or designate sub-user accounts for sub-users such as payroll administrators or office administrators. As illustrated in FIG. 3, it is also anticipated that the homecare system 1 may also create user accounts to a patient 13, a pharmacy 14, a physician 15, or other medical professionals on their respective computing devices 10, as shown in FIG. 3. Based on the type of user account created, the software application 20 requires the user to disclose predetermined personal data, generates a template for establishing a profile and allows uploading of necessary documents. The users have the option to update their profiles at a later time if needed. For one instance, the software application 30 would require each of the homecare providers 12 to disclose information including but is not limited to working eligibility, education, references, previous employment and military service experience and allow each of the homecare providers 12 to upload a resume or other supporting documents.

As a non-limiting example, the matching component 31 generates a plurality of potential matches of homecare providers 12 for each of the patients 13 which is determined based on a plurality of attributes. The plurality of attributes comprises dates, times, geographic locations, requirements of each of the patients 13 and qualifications of each of the homecare providers 12. The potential matches of the homecare providers 12 comprise the homecare providers 12 within the roster or not within the roster of each of the two or more homecare agencies 11. In a circumstance where the potential matches are the homecare providers 12 listed within the roster, the matching component 31 allows the homecare agency 11 with the roster to confirm a matched homecare provider 12 for each of the patients 13. After the matched homecare provider 12 is confirmed, the homecare agency 11 with the roster and the matched homecare provider 12 have access to view the physiological parameters of the patient 13. In another case where the potential matches are within the roster of another homecare agency 11, the revenue sharing component 32 allows the homecare providers 12 within the roster of another homecare agency 11 to accept or reject the patient 13. When the patient 13 is accepted, the revenue sharing component 32 creates an engagement record including an agreement of division of revenue between the two homecare agencies 11 and provides the two homecare agencies 11 and the homecare provider 12 accepting the patient 13 access to view the physiological parameters of each of the patient 13.

In another implementation of the invention, the revenue sharing component 32 comprises the collaborative platform. The collaborative platform is configured to allow each of the two or more homecare agencies 11 to list each of the patients 13 within their roster on the collaborative platform and specify the highest rate they are willing to accept. Relatively, the collaborative platform enables each of the two or more homecare agencies 11 to submit a proposal. The proposal includes the potential matches and rates of the homecare provider 12 for providing the homecare service to the patient 13. After viewing all the proposals, the homecare agency 12 listing the patient 13 may select the homecare agency 11 to collaborate. Thereafter, an engagement record will be generated, including an agreement of division of revenue between the two homecare agencies 11 and provides the two homecare agencies 11 and the homecare provider 12 accepting the patient access to view the physiological parameters of each of the patient.

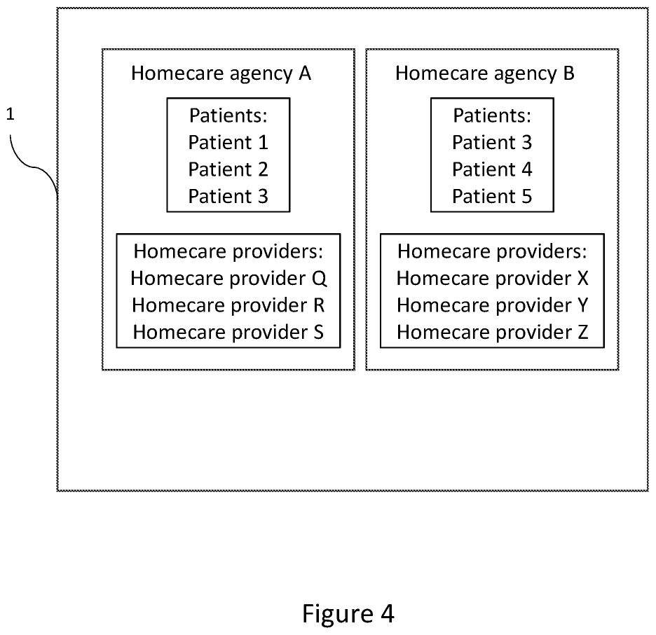

For purpose of this application the applicant will set forth examples to discuss how the homecare agencies and the homecare providers will employ the matching component and the revenue sharing component as shown in FIGS. 4 and 6.

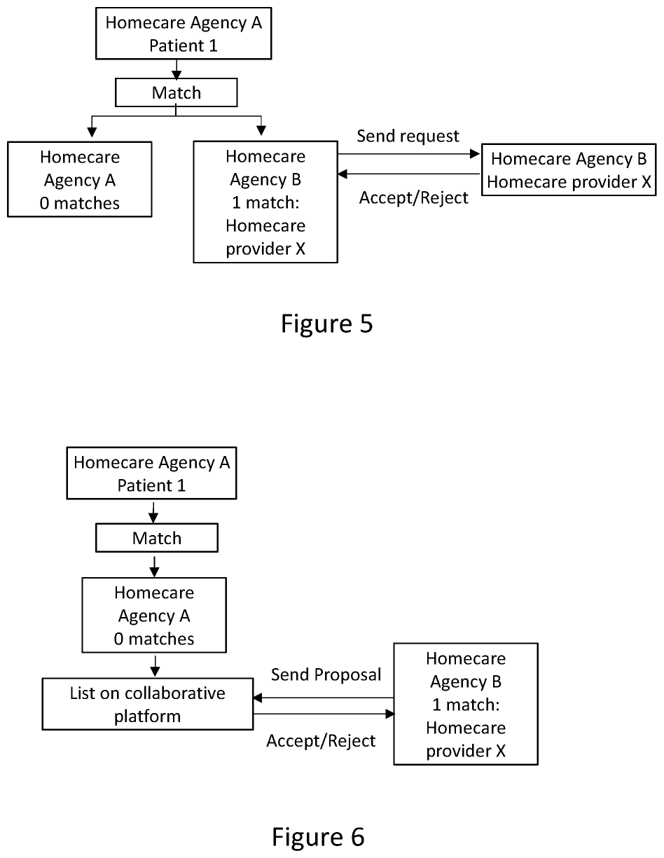

A nonlimiting example illustrated in FIG. 4, the homecare system 1 has two distinct homecare agencies 11, referred to as “homecare agency A” and “homecare agency B”, each maintains its own roster of patients and homecare providers. “Patient 1” is a patient 13 within the roster of the “homecare agency A”. The matching component of the software application generates potential matches of homecare providers for “patient 1” based on different attributes. As shown in FIG. 5, the results of the potential matches show no suitable homecare providers within the roster of “homecare agency A”, but “homecare agency B” has an appropriate homecare provider generated by the matching component, identified as “homecare provider X”. The revenue sharing component allows homecare agency A to send a request to “homecare provider X”, where “homecare provider X” from receives the request and may accept or reject the request.

In an alternative nonlimiting example as shown in FIG. 6, “homecare agency A” can list “patient 1” on the collaborative platform when no suitable potential matches of homecare providers within its roster are shown. The collaborative platform enables other homecare agencies to submit a proposal. “Homecare agency A” sends a proposal of “homecare provider X” and the rates for homecare provider X to perform the homecare service for “homecare agency A”. “Homecare agency A” may accept or reject the proposal. Other homecare agencies may also submit their proposals to “Homecare agency A”, and “Homecare agency A” may select the desired homecare provider.

Upon mutual confirmation of the match, the revenue sharing component creates an engagement record for “homecare agency A” and “homecare agency B”. Additionally, the revenue sharing component provides the two homecare agencies and “homecare provider X” access to view the physiological parameters of “patient 1”.

The recruiting component 33 enables the homecare agencies 11 to recruit homecare providers 12 and allows the homecare providers 12 to discover and apply for employment opportunities with these homecare agencies 11. Each of the homecare providers 12 may execute one or more actions, which include, but are not limited to viewing the homecare agencies 11 within a predetermined distance or service zone, reviewing the profile of the homecare agencies 11, submitting applications to the homecare agencies 11, uploading, reviewing, or signing one or more documents, and receiving notifications. The recruiting component 33 allows each of the homecare agencies 11 to execute one or more actions, which include, but are not limited to, reviewing the profile of the homecare providers 12, viewing the homecare providers 12 within a predetermined distance or service zone of the homecare agency 11, receiving and accepting applications from the homecare provider 12, uploading, reviewing, or signing one or more documents, tracking onboarding status, and receiving notifications. The homecare providers 11 may filter to view the homecare agencies 11 that provide homecare services within a predetermined distance or service zone according to physical addresses or zip codes. It is anticipated that the recruiting component 33 may be connected to the GPS receiver 103 of each of the plurality of computing devices 10, such that the homecare providers 12 may filter to view the homecare agencies 11 that provide homecare services within a predetermined distance according to the respective locations determined by their communication device 10. Subsequently, each of the homecare providers 11 may submit their profile to one or more of the homecare agencies 11 as an application. Relatively, each of the two or more homecare agencies 11 may view the homecare providers 12 within their service zone and send an offer. The recruiting component 32 generates notifications once the application or offer was sent, and then the homecare agency 11 may select to accept or reject the application, or the homecare provider 12 may choose to accept or reject the offer. Once the application was accepted by the homecare provider 12 or the offer was accepted by the homecare agency 11, the recruiting component generates an onboarding procedure for the homecare provider 12 to complete. The onboarding procedure of the recruiting component 32 requires the homecare provider 12 to complete required documents by generating online forms, providing fillable forms, or allowing document uploading.

The mapping component 34 provides an interactive map that interfaces with one or more third-party geospatial mapping applications. The interactive map denotes the geographic location for each of the patients 13, and the potential matches of the homecare providers 12 generated by the matching component 31. Each of the two or more homecare agencies 11 may view the geographic location of the potential matches in relation to the patient's geographic location, and subsequently send a request to the desired potential matches. It is also anticipated that the mapping component 34 is connected to the GPS receiver 103 of each of the computing devices 10, such that each of the two or more homecare agencies 11 may view its respective locations in relation to the geographic locations of each of the patient 13 and the potential matches.

The referring component 35 facilitates the process of intaking patient referrals from various sources, such as physicians, pharmacies, clinics, other medical institutions, professionals, or insurance companies. Each of the homecare agencies 11 may execute one or more actions, which include, but are not limited to creating, viewing, editing the patient referrals. Each of the two or more homecare agencies may also create a profile for each of the sources.

Once the matched homecare provider 12 is confirmed, the scheduling component 36 enables each of two or more homecare agencies 11 to create a number of visits for each of the patients 13 and input frequency of the visits which is determined in accordance with the specific requirements of each of the individual patient 13. The scheduling component 36 provides a calendar view or a list view showing past and future visits. Each of the two or more homecare agencies 11 may also execute other actions, which include, but are not limited to, updating, canceling the visits, reassigning a homecare provider for the visits, tracking status of the visit, reviewing doctor's order, patient's profile and infusion notes for the visits, viewing visit history, verifying the last visits, discharging the patients, and extending the visits. Each of the homecare providers 12 may also execute other actions, which include, but are not limited to, updating, canceling the visits, reviewing doctor's order, patient's profile, creating infusion notes for the visits, viewing visit history, indicating the last visit.

The medical record component 37 enables the two or more homecare providers 11 or the plurality of homecare providers 12 to upload, view, or generate comprehensive documents related to each of the individual patient 13, which include, but are not limited to patient's medical history, diagnosis, doctor's order, plan of treatment, treatment report, infusion notes, progress notes, test results, physiological parameters, lab orders and supplies, discharge report, and other records related to each of the patient's healthcare. The medical record component 37 restricts the medical records to only be viewable by the homecare agency 11 with the roster of the patient 13, the matched homecare provider 12 and the homecare agency 11 collaborating.

It is also anticipated that the software system also provides a plurality of other functions including notetaking, inputting timesheets, and messaging to optimize administrative task or management.

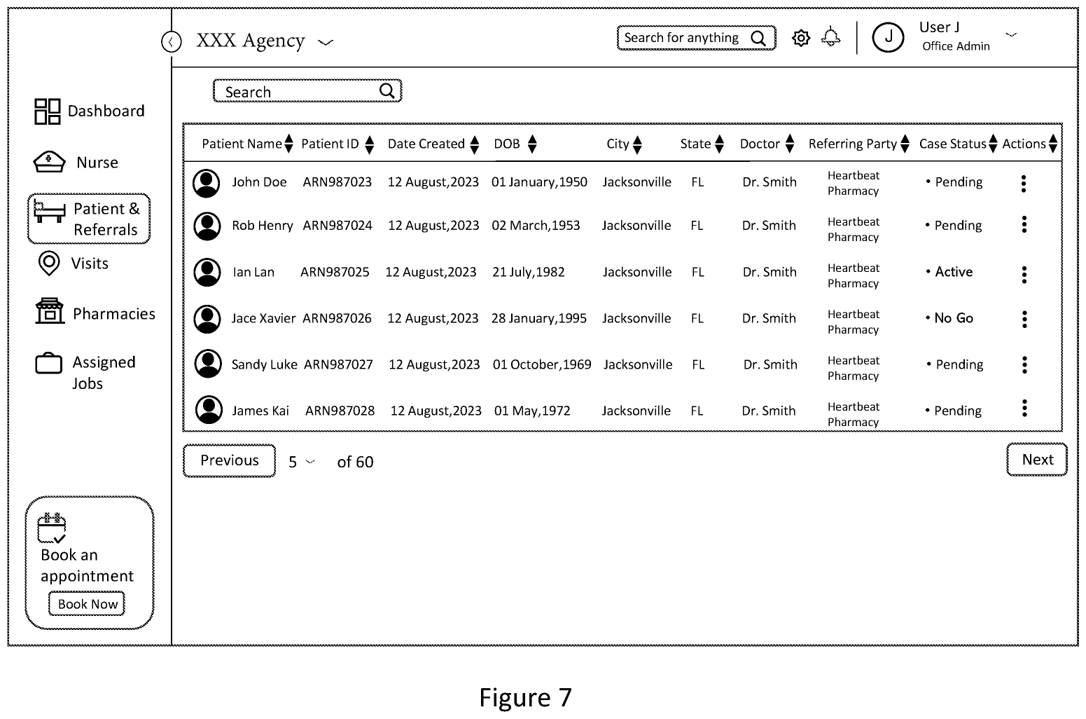

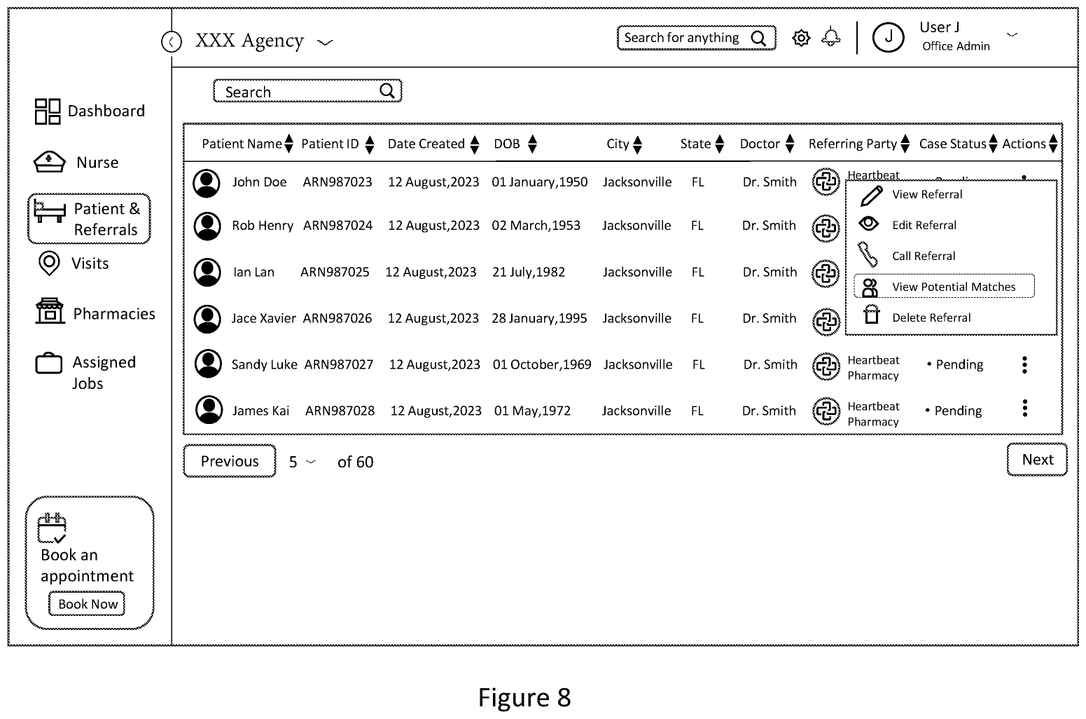

FIGS. 7 and 8 illustrate exemplary pages showing the patients 13 within the roster of one of the homecare agencies. The homecare agency 11 may search, view, edit, call, delete or view potential matches of each of the patient.

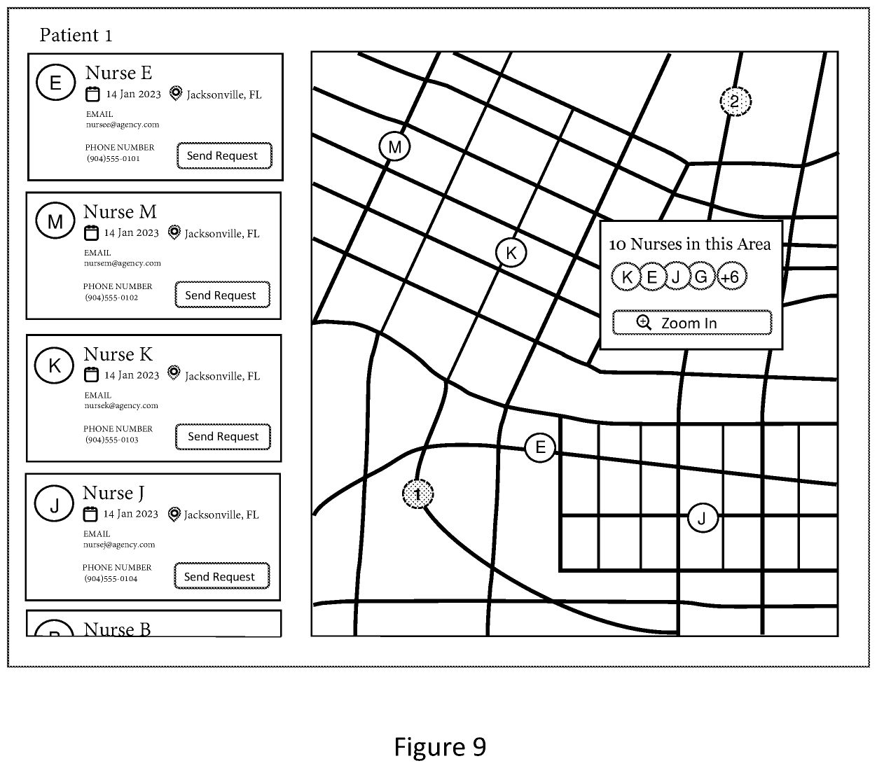

FIG. 9 illustrates an exemplary page showing the interactive map of the mapping component 34. The interactive map shows the respective geographic locations of the potential matches for “patient 1” and allows the homecare agency 11 of “patient 1” to send requests to the desired homecare providers 12.

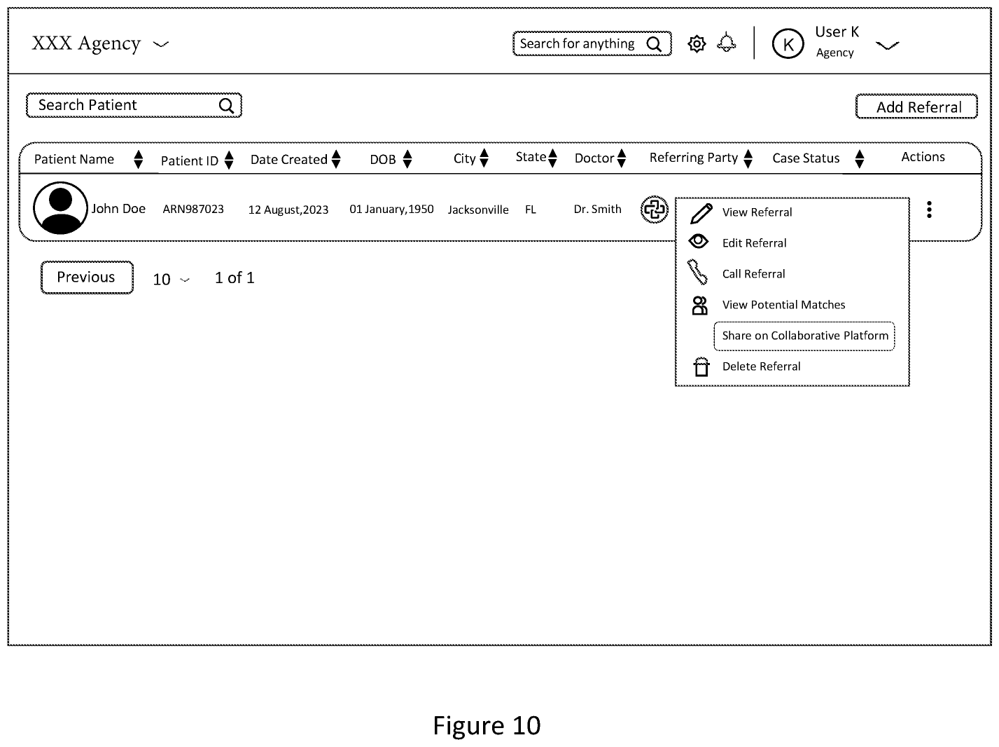

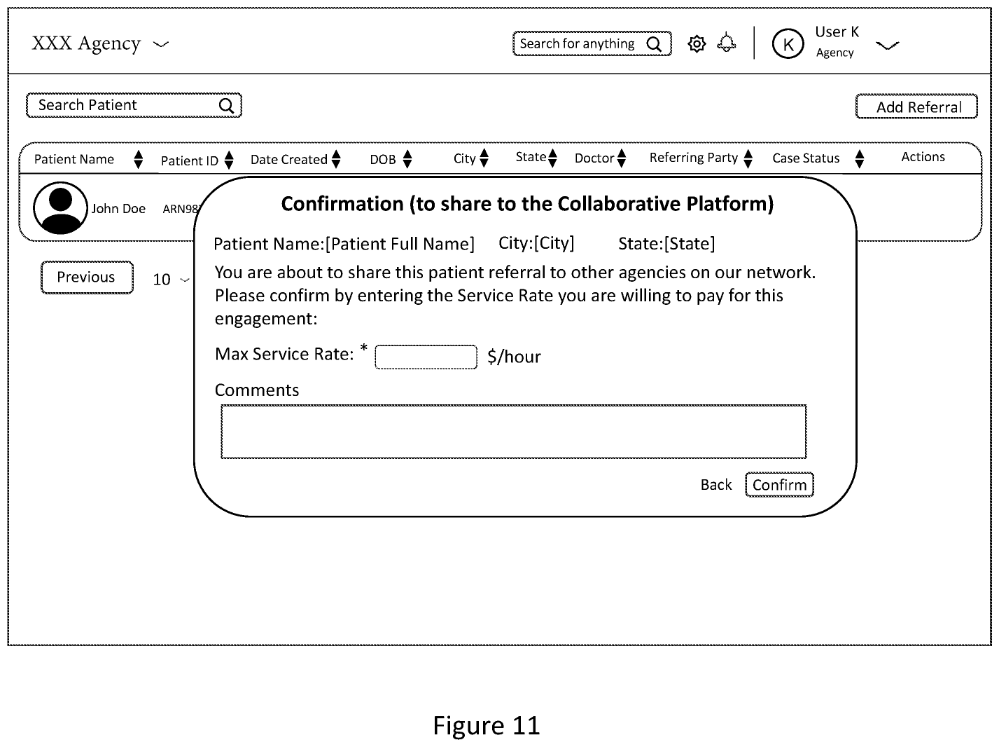

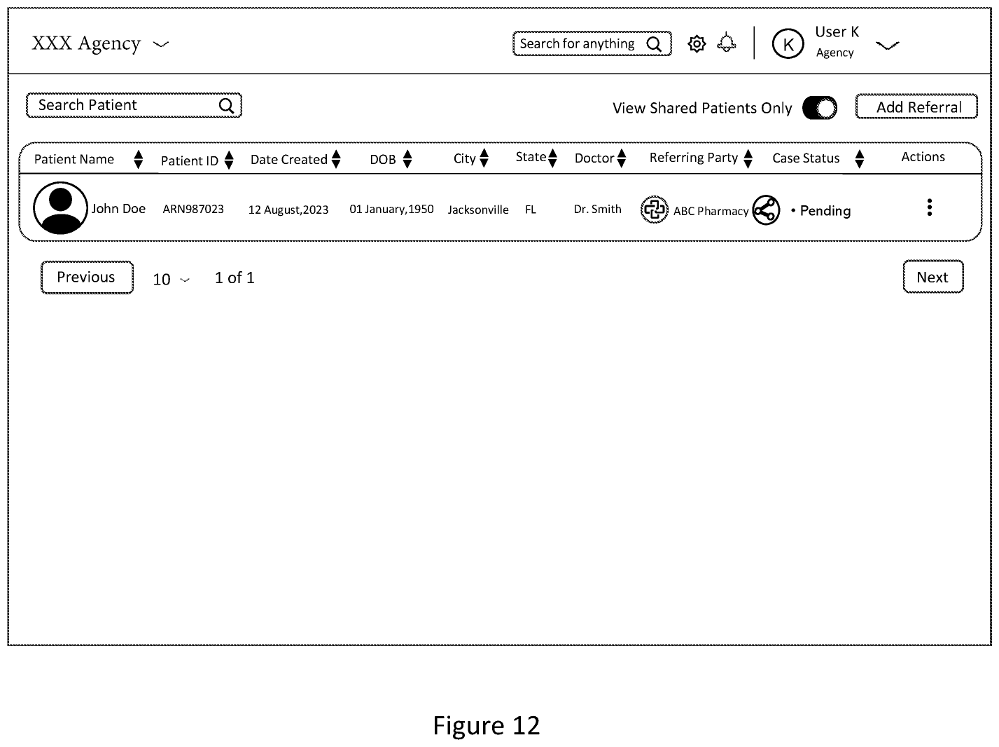

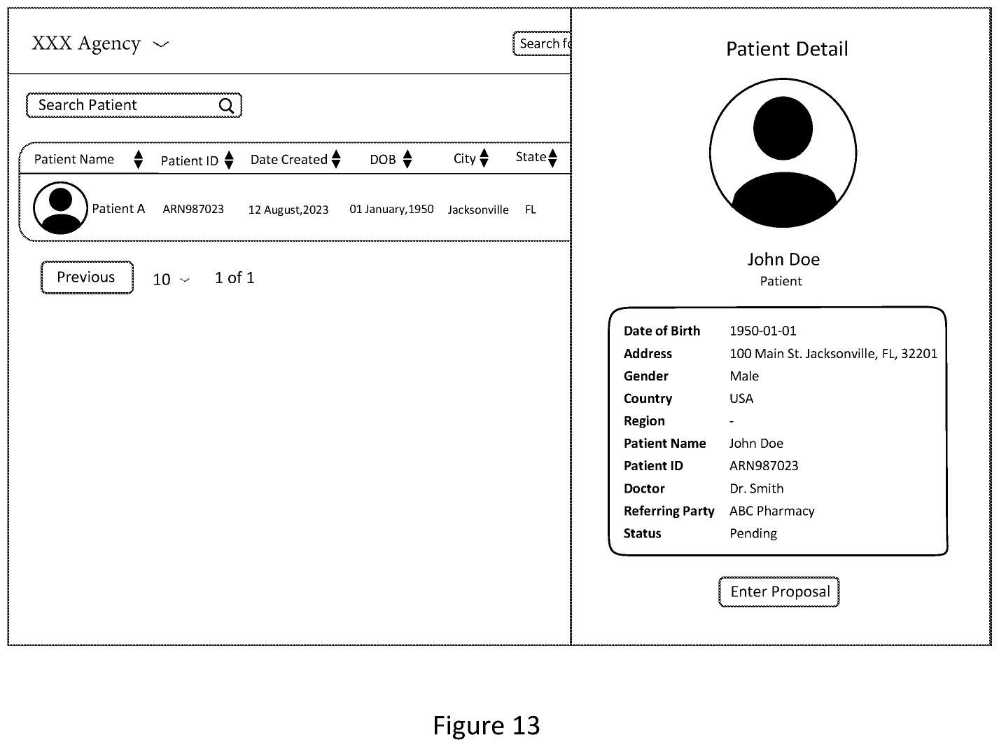

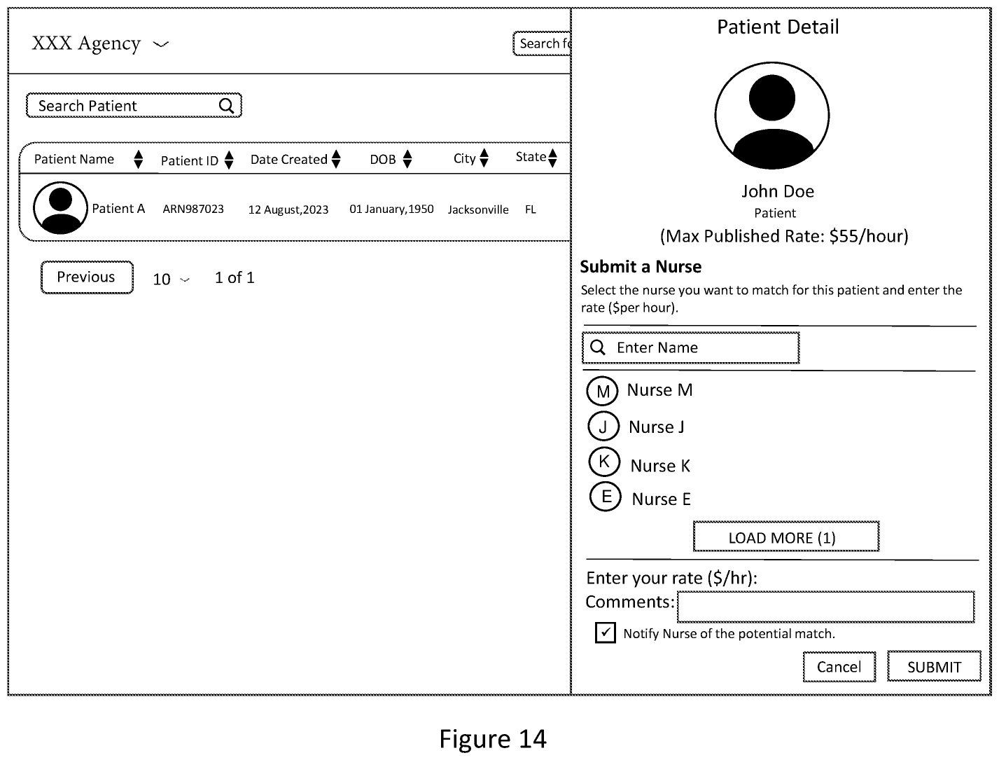

FIGS. 10 to 14 illustrate exemplary pages demonstrating the revenue sharing component 32. FIGS. 10 to 11 shows the homecare agency 11 may list a patient to the collaborative platform and specify the highest rate they are willing to accept. FIG. 12 depicts that the homecare agency 11 may filter to view only shared patient. FIG. 13 to 14 depict the process in which the homecare agency may submit a proposal that includes a potentially matched homecare provider and the corresponding rate.

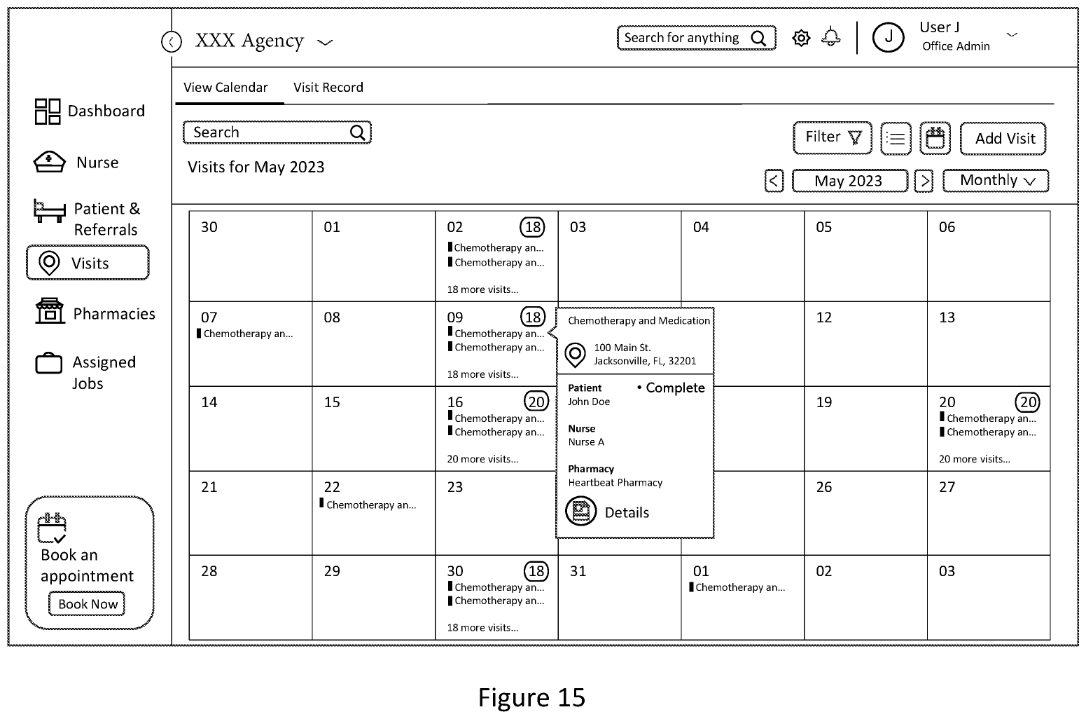

FIG. 15 illustrates an exemplary page showing the calendar view provided by the scheduling component 36. The calendar enables the homecare agencies 11 and the homecare providers 12 to conveniently access records of past and future visits.

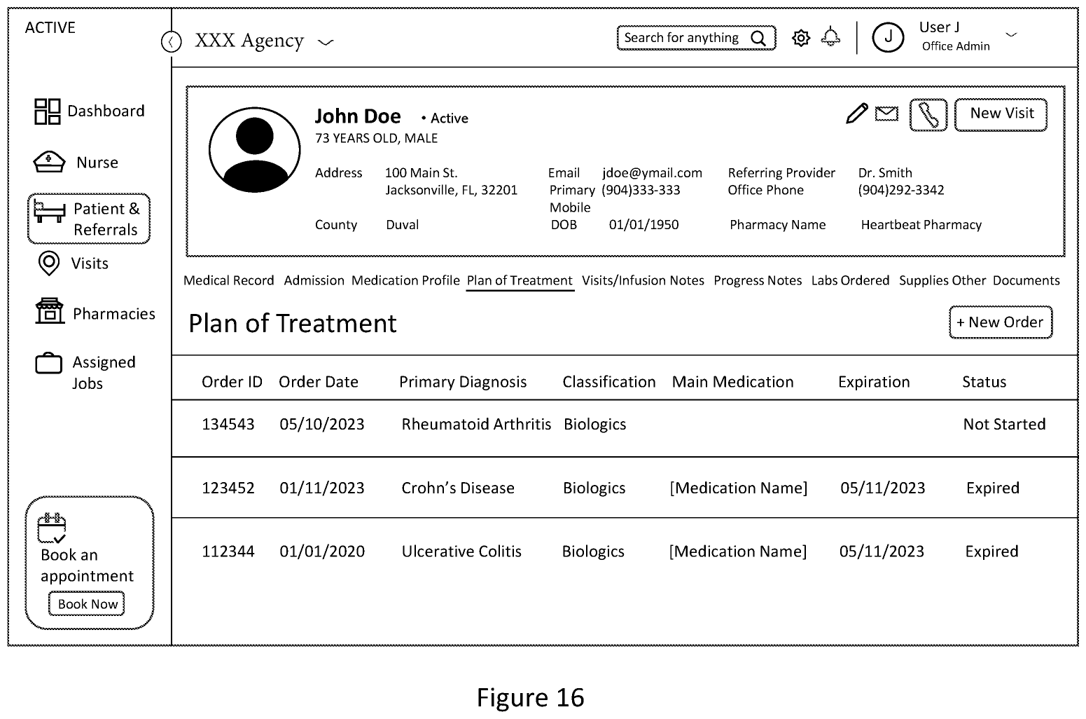

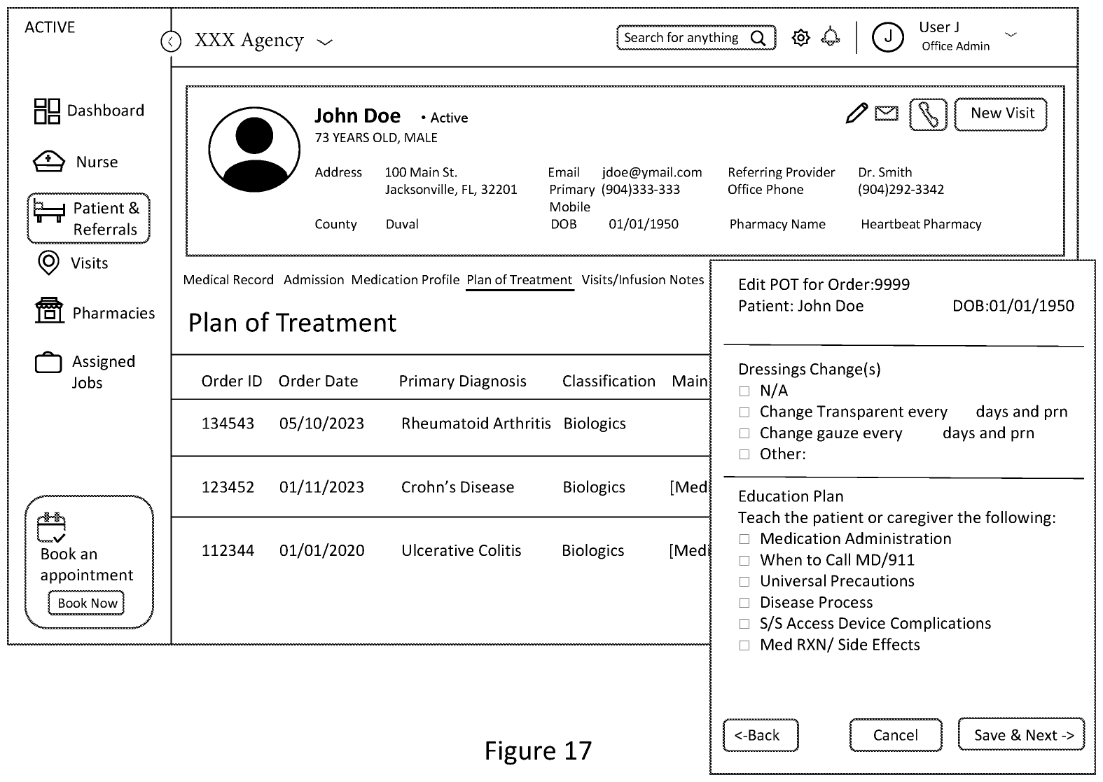

FIGS. 16 to 17 illustrate exemplary pages of the medical record component 37, specifically the plan of treatment.

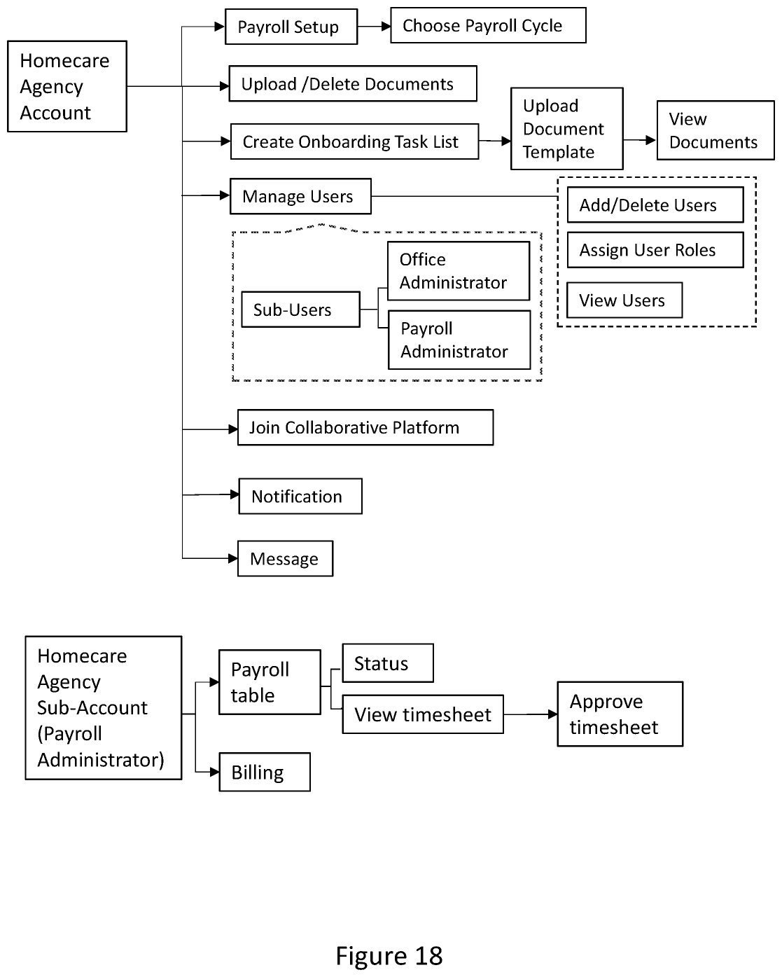

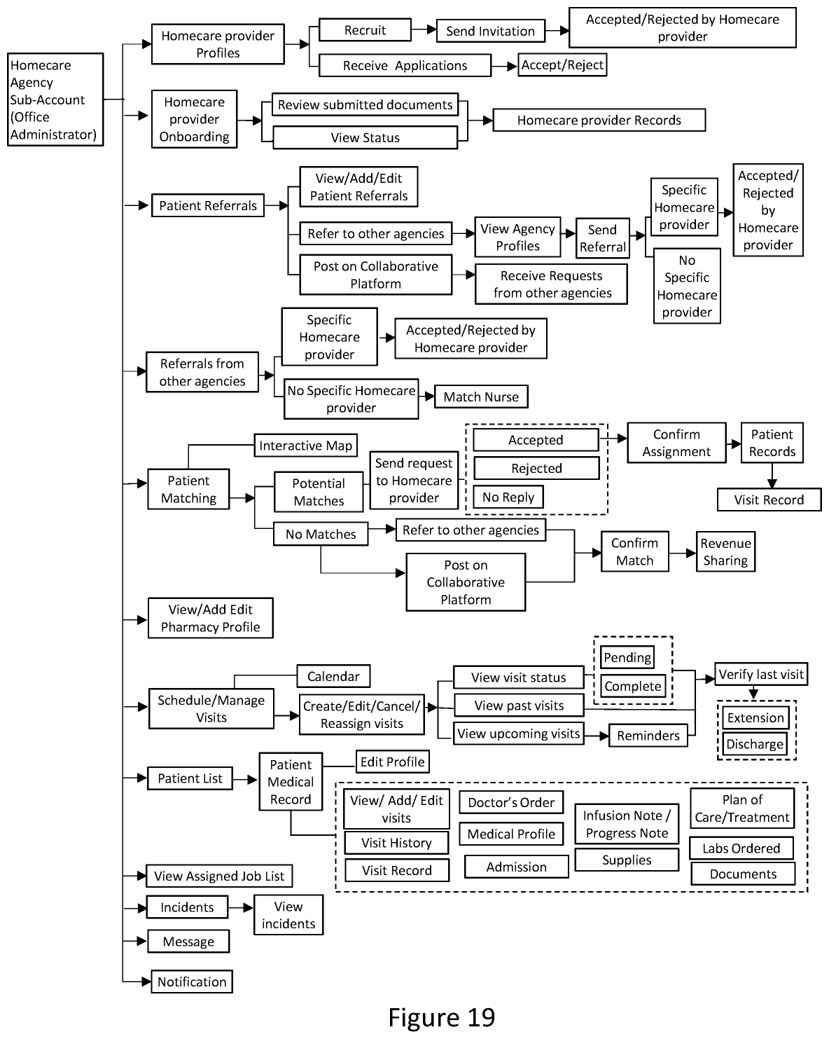

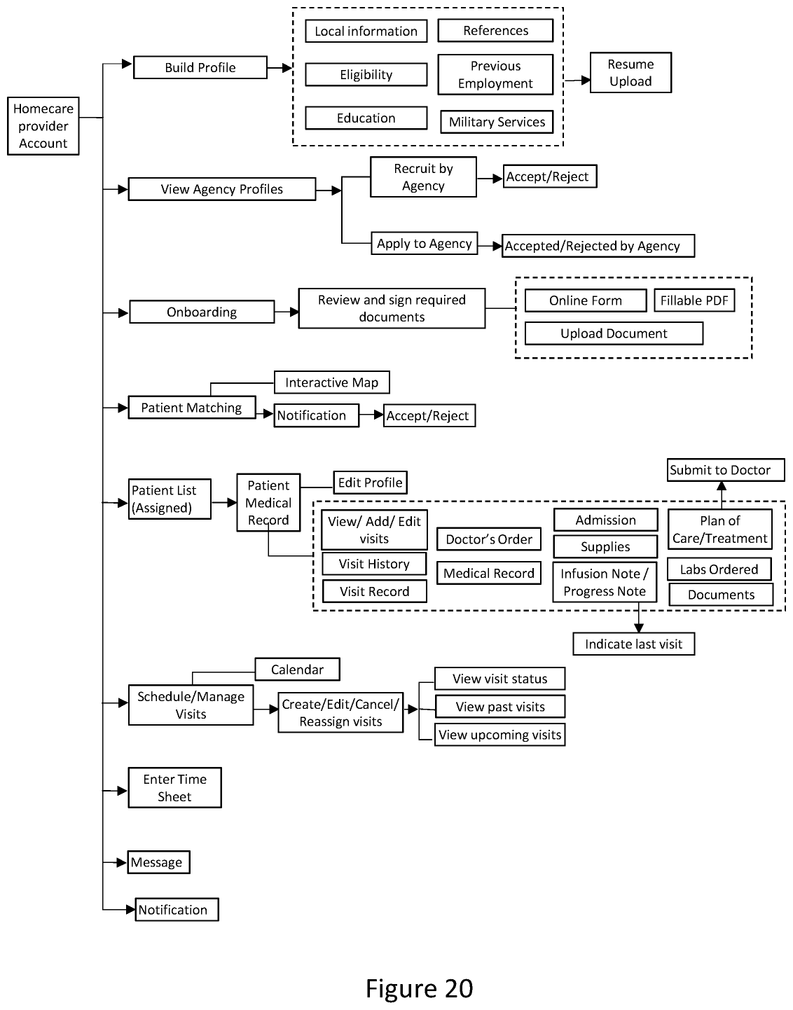

FIGS. 18 to 20 depicts association between a variety of features in perspectives of the homecare agencies 11 and homecare providers 12.

While the exemplary embodiment of the present disclosure has been disclosed, certain modifications may be made by those skilled in the art to modify the invention without departing from the spirit of the invention.

1. A system for facilitating collaboration in healthcare comprising:
a. a network;
wherein the network includes a plurality of wirelessly connected end nodes;
b. two or more homecare agencies, a plurality of homecare providers and a plurality of patients;
wherein each of the two or more homecare agencies has a roster of patients and homecare providers;
c. a plurality of computing devices associated with each of the two or more homecare agencies and the plurality of homecare providers respectively;
wherein each of the computing devices is connected to the network;
d. one or more servers;
wherein the one or more servers are communicated with the network;
e. a database;
wherein the database is stored on the one or more servers;
f. one or more monitoring devices;
wherein each of the one or more monitoring devices are configured to monitor physiological parameters of each of the plurality of patients;
wherein the one or more monitoring devices are connected to the network wirelessly;
wherein the physiological parameters are configured to be received remotely by the plurality of computing devices; and
g. a software application accessible from the plurality of computing devices;
wherein the software application is communicated with the network and synced to the database;
wherein the software application further comprises a matching component;
wherein the matching component generates for each of the plurality of patients, a plurality of potential matches of the plurality homecare providers;
wherein the potential matches are determined based on a plurality of attributes;
wherein the plurality of attributes comprises dates, times, geographic locations, requirements of each of the plurality of patients and qualifications of each of the plurality of homecare providers;
wherein the matching component determines for each of the two or more homecare agencies, whether the homecare care providers of the potential matches are listed on the roster of each of the two or more homecare agencies or listed on the roster of another homecare agency;
in response to the potential matches being listed on the roster, the matching component provides a confirmation option to the homecare agency;
in response to the confirmation option, the matching component provides the homecare agency having the roster and the matched homecare provider access to view the physiological parameters of the patient;
a revenue sharing component;
in response to the potential matches listed on the roster of another homecare agency, the revenue sharing component provides an accept option or a reject option to the homecare providers listed on the roster of another agency;
wherein the revenue sharing component identifies the accept option or the reject option being selected;
in response to identifying the accept option being selected, the revenue sharing component creates an engagement record including an agreement of division of revenue between the two homecare agencies and provides the two homecare agencies and the homecare provider accepting the patient access to view the physiological parameters of the patient.

2. The system as described in claim 1, wherein the software application provides user accounts to each of the two or more homecare agencies and the plurality of homecare providers.

3. The system as described in claim 1, wherein each of the computing devices further comprises a camera; wherein the camera is configured to capture two-dimensional codes on medication or supplies for each of the plurality of patients and identify medical data from the two-dimensional codes.

4. The system as described in claim 3, wherein the matching component provides the homecare agency having the roster and the matched homecare provider access to view the medical data of the patient; and wherein the revenue sharing component provides the two homecare agencies and the homecare provider accepting the patient access to view the medical data of the patient.

5. The system as described in claim 1, wherein the software application further comprises a recruiting component;
wherein the recruiting component displays the homecare agencies providing homecare services in a predetermined area for each of the homecare providers; and
wherein the recruiting component transmits applications from each of the homecare providers to each of the homecare agencies.

6. The system as described in claim 5, wherein the recruiting component identifies whether each of the two or more homecare agencies accepts or rejects the applications; in response to identifying accepting the application, the recruiting component generates required documents to each of the homecare providers.

7. The system as described in claim 1, wherein the software application further comprises a mapping component;
wherein the mapping component provides an interactive map; and
wherein the mapping component denotes the geographic locations of each of the plurality of patients and the potential matches.

8. The system as described in claim 1, wherein the software application further comprises a referring component configured to create and manage profile of each of the plurality of patients.

9. The system as described in claim 1, wherein the software application further comprises a scheduling component;
wherein the scheduling component is configured to provide each of the two or more homecare agencies and the homecare providers to create a number of visits for each of the plurality of patients and input frequency of the visits.

10. The system as described in claim 1, wherein the software application further comprises a medical record component configured to provide each of the two or more homecare agencies and the plurality of homecare providers to view, upload or edit medical records of each of the plurality of patients.

11. A system for facilitating collaboration in healthcare comprising:
a. a network;
wherein the network includes a plurality of wirelessly connected end nodes;
b. two or more homecare agencies, a plurality of homecare providers and a plurality of patients;
wherein each of the two or more homecare agencies has a roster of patients and homecare providers;
c. a plurality of computing devices associated with each of the two or more homecare agencies and the plurality of homecare providers respectively;
wherein each of the computing devices is connected to the network;
d. one or more servers;
wherein the one or more servers are communicated with the network;
e. a database;
wherein the database is stored on the one or more servers;
f. one or more monitoring devices;
wherein each of the one or more monitoring devices are configured to monitor physiological parameters of each of the plurality of patients;
wherein the one or more monitoring devices are connected to the network wirelessly;
wherein the physiological parameters are received by the plurality of computing devices; and
g. a software application accessible from the plurality of computing devices;
wherein the software application is communicated with the network and synced to the database;
wherein the software application provides user accounts to each of the two or more homecare agencies and the plurality of homecare providers;
wherein the software application further comprises:
a matching component;
wherein the matching component generates for each of the plurality of patients, a plurality of potential matches of the plurality homecare providers;
wherein the potential matches are determined based on a plurality of attributes;
wherein the plurality of attributes comprises dates, times, geographic locations, requirements of each of the plurality of patients and qualifications of each of the homecare providers;
wherein the matching component determines for each of the homecare agencies, whether the homecare care providers of the potential matches are with the roster of each of the homecare agencies;
in response to the potential matches within the roster, the matching component provides a confirmation option to the homecare agency;
in response to the confirmation option, the matching component provides the homecare agency having the roster and the matched homecare provider access to view the physiological parameters of the patient;
a revenue sharing component;
wherein the revenue sharing component comprises a collaborative platform;
wherein the collaborative platform is configured to provide each of the two or more homecare agencies to list each of the plurality of patients within the roster on the collaborative platform and enable each of the two or more homecare agencies to submit a proposal;
wherein the proposal comprises the potential matches of the homecare providers and corresponding rates;
wherein the revenue sharing component identifies the homecare provider selected to collaborate;
in response to identifying the homecare provider selected, the revenue sharing component creates an engagement record including an agreement of division of revenue between the two homecare agencies collaborating and provides the two homecare agencies and the homecare provider collaborating access to view the physiological parameters of the patient.

12. The system as described in claim 11, wherein the software application provides user accounts to each of the two or more homecare agencies and the plurality of homecare providers.

13. The system as described in claim 11, wherein each of the computing devices further comprises a camera; wherein the camera is configured to capture two-dimensional codes on medication or supplies for each of the plurality of patients and identify medical data from the two-dimensional codes.

14. The system as described in claim 13, wherein the matching component provides the homecare agency having the roster and the matched homecare provider access to view the medical data of the patient; and wherein the revenue sharing component provides the two homecare agencies and the homecare provider accepting the patient access to view the medical data of each patient of the plurality of patients.

15. The system as described in claim 11, wherein the software application further comprises a recruiting component;
wherein the recruiting component displays the homecare agencies providing homecare services in a predetermined area for each of the homecare providers; and
wherein the recruiting component transmits applications from each of the homecare providers to each of the homecare agencies.

16. The system as described in claim 15, wherein the recruiting component identifies whether each of the two or more homecare agencies accepts or rejects the applications; in response to identifying accepting the application, the recruiting component generates required documents to each of the homecare provider.

17. The system as described in claim 11, wherein the software application further comprises a mapping component;
wherein the mapping component provides an interactive map; and
wherein the mapping component denotes the geographic locations of each of the plurality of patient and the potential matches.

18. The system as described in claim 11, wherein the software application further comprises a referring component configured to create and manage profile of each of the plurality of patients.

19. The system as described in claim 11, wherein the software application further comprises a scheduling component;
wherein the scheduling component is configured to provide each of the two or more homecare agencies and the homecare providers to create a number of visits for each of the plurality of patients and input frequency of the visits.

20. The system as described in claim 11, wherein the software application further comprises a medical record component configured to provide each of the two or more homecare agencies and the plurality of homecare providers to view, upload or edit medical records of each of the plurality of patients.
